(19) United States
(12) Patent Application Publication
(10) Pub. No.: US 2020/0017483 A1
(43) Pub. Date: Jan. 16, 2020

(54) A PROCESS FOR THE PREPARATION OF D-GLUCITOL, 1,5-ANHYDRO-1-C-[4-CHLORO-3-[[4- [[(3S)-TETRAHYDRO-3-FURANYL]OXY]PHENYL]METHYL]PHENYL]-, ( 1 S)

(71) Applicants: Srinivasan THIRUMALAI RAJAN, Hyderabad, Telangana (IN); Sajja ESWARAIAH, Hyderabad, Telangana (IN); Gogulapati VENKATA PANAKALA RAO, Hyderabad, Telangana (IN); Sagyam RAJESHWAR REDDY, Hyderabad, Telangana (IN); Shaik MOHAMMAD RAFEE, Hyderabad, Telangana (IN); Rangineni SRINIVASULU, Hyderabad, Telangana (IN); MSN LABORATORIES PRIVATE LIMITED, R&D CENTER, Hyderabad, Telangana (IN)

(72) Inventors: Thirumalai Rajan SRINIVASAN, Hyderabad (IN); Eswaraiah SAJJA, Hyderabad (IN); Venkata Panakala Rao GOGULAPATI, Hyderabad (IN); Rajeshwar Reddy SAGYAM, Hyderabad (IN); Mohammad Rafee SHAIK, Hyderabad (IN); Srinivasuslu RANGINENI, Hyderabad (IN)

(73) Assignee: MSN LABORATORIES PRIVATE LIMITED, R&D CENTER, Hyderabad, Telangana (IN)

(21) Appl. No.: 16/492,933

(22) PCT Filed: Mar. 12, 2018
(86) PCT No.: PCT/IN2018/000019

§ 371 Date: Sep. 10, 2019

(30) Foreign Application Priority Data
Mar. 10, 2017 (IN) 201741008395

(51) Int. Cl. C07D 407/12

(52) U.S. Cl. CPC C07D 407/12

(57) ABSTRACT

The present invention relates to a process for the preparation of D-glucitol, 1,5-anhydro-1-C-[4-chloro-3-[[4-[[(3S)-tetrahydro-3-furanyl)oxy)phenyl] methyl]phenyl]-, (IS) formula-1.

### RELATED APPLICATION

This patent application claims the benefit of priority of our Indian patent application number 201741008395 filed on 10 Mar. 2017 which is incorporated herein by reference.

### FIELD OF THE INVENTION

The present invention relates to a process for the preparation of D-glucitol, 1,5-anhydro-1-C-[4-chloro-3-[[4-[[(3S)-tetrahydro-3-furanyl]oxy]phenyl]methyl] phenyl]-, (1S), which is represented by the following formula-1

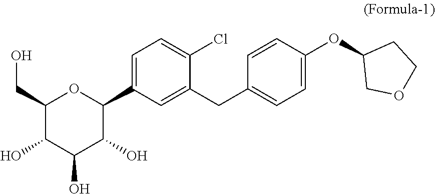 [SMILES: OC[C@H]1O[C@@H](c2ccc(Cl)c(Cc3ccc(O[C@H]4CCOC4)cc3)c2)[C@H](O)[C@@H](O)[C@@H]1O]

### BACKGROUND OF THE INVENTION

D-Glucitol, 1,5-anhydro-1-C-[4-chloro-3-[[4-[[(3S)-tetrahydro-3-furanyl]oxy]phenyl]methyl] phenyl]-, (1S) also known as “Empagliflozin” is sodium-glucose co-transporter 2 (SGLT2) and this drug product indicated as an adjunct to diet and exercise to improve glycemic control in adults with type 2 diabetes mellitus. It is marketed by Boehringer under the trade name Jardiance® and it is available in 10 mg and 25 mg strengths as tablet dosage forms.

U.S. Pat. No. 7,579,449 describes Empagliflozin product and also a process for the preparation thereof comprising reaction of 4-bromo-1-chloro-2-(4-methoxybenzyl)-benzene with tribromoborane in dichloromethane to produce 4-(5-bromo-2-chloro-benzyl)-phenol which is reacted with t-butyl dimethyl silyl chloride in dichloromethane in presence of triethylamine and dimethylaminopyridine to get [4-(5-bromo-2-chloro-benzyl)-phenoxy]-tert-butyldimethyl-silane which is further reacted with n-BuLi in tetrahydrofuran followed by coupling with 2,3,4,6-tetrakis-O-(trimethylsilyl)-D-glucopyranone. The resulting reaction mixture is treated with methanesulfonic acid (MsOH) in methanol followed by reduction with triethylsilylhydride and boron trifluoride etherate and acylated with acetic anhydride/pyridine in dichloromethane followed by treating with KOH in methanol to produce (2S, 3R, 4R, 5S, 6R)-2-(4-chloro-3-(4-hydroxybenzyl)phenyl)-6-(hydroxymethyl) tetrahydro-2H-pyran-3,4,5-triol (pentahydroxy intermediate). This pentahydroxy intermediate is reacted with tetrahydrofuran-3-yl (S)-toluene-4-sulphonate in presence of cesium carbonate (Cs₂CO₃) in dimethylformamide (DMF) to produce the Empagliflozin of formula-1.

The major disadvantage of the above process is the difficulty in avoiding formation/separation of (R)-isomer of Empagliflozin which is formed on reaction of tetrahydrofuran-3-yl (S)-toluene-4-sulphonate intermediate compound with pentahydroxy intermediate. This is due to use of tetrahydrofuran-3-yl (S)-toluene-4-sulphonate intermediate which is exists as a residue nature the contamination of the R-isomer. Hence, this process requires multiple purification methods to remove the R-isomer of Empagliflozin which led to increasing the cost of the product. Therefore, the above process is not viable for industrial scale preparation.

The above prior art process also produces very low yield of Empagliflozin on reaction of pentahydroxy intermediate with tetrahydrofuran-3-yl (S)-toluene-4-sulphonate. Further, it also involves the use of hazardous BBr₃ which reacts violently and decomposes to toxic compounds when it comes in contact with moisture.

U.S. Pat. No. 7,772,191 discloses a process for the preparation of Empagliflozin of formula-1 comprising reaction of 2,3,4,6-tetrakis-O-(trimethylsilyl)-D-glucopyranone with (S)-4-bromo-1-chloro-2-(4-tetrahydrofuran-3-yloxy-benzyl)-benzene in presence of t-BuLi in pentane and methanesulfonic acid (MsOH) in methanol to produce 1-chloro-4-(1-methoxy-D-glucopyranos-1-yl)-2-(4-(S)-tetrahydrofuran-3-yloxy-benzyl)benzene which is reduced with triethylsilane in presence of BF₃ etherate and treatment with aceticanhydride to produce the 1-chloro-4-(2,3,4,6-tetra-O-acteyl-D-glucopyranos-1-yl)-2-(4-(S)-tetrahydrofuran-3-yloxy-benzyl)-benzene (tetra acyl protected Empagliflozin) which is further hydrolyzed to produce the Empagliflozin of formula-1.

U.S. Pat. No. 7,772,191 also discloses another process for the preparation of Empagliflozin of formula-1 comprising reaction of 2,3,4,6-tetrakis-O-(trimethylsilyl)-D-glucopyranone with (S)-4-bromo-1-chloro-2-(4-tetrahydrofuran-3-yloxy-benzyl)-benzene in presence of BuMgCl in THF, BuLi in hexane and then reacting with MeSO₃H in MeOH and then reduced with triethylsilane in presence of BF₃ etherate to produce the Empagliflozin of formula-1.

The major disadvantage with the above prior art process is preparation and isolation of tetra acetyl protected Empagliflozin which requires additional step of hydrolysis of the same to produce the Empagliflozin. Another process in the prior art involves the use of BuMgCl which is extremely inflammable and will cause flash fire or ignite explosively. Hence, the handling of BuMgCl is very dangerous and will not be suitable for industrial scale preparations.

In view of the above facts, there still remains a need to develop an alternate, simple and improved process for the preparation of Empagliflozin of formula-1 with high chemical and enantiomerical purity and also applicable for multi-kilogram production. The process of the present invention is inexpensive, environmental-friendly having straight forward workups, rendering it amenable to the large-scale production of formula-1 with high yield.

The present inventors also worked to reduce the formation of unrequired R-isomer of Empagliflozin in the final coupling reaction to get pure Empagliflozin. For that they conducted several experiments by using different type of protected compounds of formula-2, solvents, but none of them provides the pure Empagliflozin with high yield. Unexpectedly, they controlled the R-isomer within the limits when the coupling reaction carried out with pure solid compound of (R)-tetrahydrofuran-3-yl 4-nitrobenzenesulfonate in a suitable organic solvents. Preferably the suitable organic solvent is n-butanol.

### ADVANTAGES OF THE PRESENT INVENTION

Less number of synthetic steps to prepare pure Empagliflozin, which leads to the consumption of less number of solvents, reagents, intermediates thereby reducing the formation of lesser pollutants making it eco-friendly and economically viable.
Reducing the lesser number of steps will decrease the time cycle thereby decreasing the man power spent, decreasing consumption of power and infrastructure etc. which brings down the cost drastically.
Controlling the formation of unwanted R-isomer of Empagliflozin in final stage which leads to increasing the yield and purity of the final product.

### BRIEF DESCRIPTION OF THE INVENTION

The first aspect of the present invention is to provide a process for the preparation of D-glucitol, 1,5-anhydro-1-C-[4-chloro-3-[[4-[[(3S)-tetrahydro-3-furanyl]oxy] phenyl]methyl]phenyl]-, (1S) of formula-1.

The second aspect of the present invention is to provide a novel crystalline form of D-glucitol, 1,5-anhydro-1-C-[4-chloro-3-[[4-[[(3S)-tetrahydro-3-furanyl]oxy] phenyl]methyl]phenyl]-, (1S) (hereinafter designated as “Form-S”).

The third aspect of the present invention is to provide a process for the preparation of crystalline Form-S of D-glucitol, 1,5-anhydro-1-C-[4-chloro-3-[[4-[[(3S)-tetrahydro-3-furanyl]oxy] phenyl]methyl] phenyl]-, (1S).

The fourth aspect of the present invention provides a process for the preparation of stable amorphous form of D-glucitol, 1,5-anhydro-1-C-[4-chloro-3-[[4-[[(3S)-tetrahydro-3-furanyl]oxy] phenyl]methyl] phenyl]-, (1S).

### BRIEF DESCRIPTION OF THE DRAWINGS

FIG. 1: Illustrates the Powdered X-Ray Diffraction (PXRD) Pattern of crystalline Form-S of Empagliflozin.

FIG. 2: Illustrates the PXRD pattern of stable amorphous form of Empagliflozin obtained according to the examples 12, 13, 14 and 15.

### DETAILED DESCRIPTION OF THE INVENTION

The present invention relates to a process for the preparation of D-glucitol, 1,5-anhydro-1-C-[4-chloro-3-[[4-[[(3S)-tetrahydro-3-furanyl]oxy]phenyl]methyl] phenyl]-, (1S) compound of formula-1.

The term “suitable solvent” used in the present invention refers to “hydrocarbon solvents” such as n-hexane, n-heptane, cyclohexane, benzene, toluene, pentane, cycloheptane, methylcyclohexane, ethyl benzene, m-, o-, or p-xylene or naphthalene and the like; “ether solvents” such as dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, methyl tert-butyl ether, 1,2-dimethoxy ethane and the like; “ester solvents” such as methyl acetate, ethyl acetate, isopropyl acetate, n-butyl acetate and the like; “polar-aprotic solvents such as dimethylacetamide (DMA), dimethylformamide (DMF), dimethylsulfoxide (DMSO), N-methylpyrrolidone (NMP) and the like; “chloro solvents” such as dichloromethane, dichloroethane, chloroform, carbon tetrachloride and the like; “ketone solvents” such as acetone, methyl ethyl ketone, pentanone, methyl isobutylketone and the like; “nitrile solvents” such as acetonitrile, propionitrile, isobutyronitrile and the like; “alcohol solvents” such as methanol, ethanol, n-propanol, isopropanol, n-butanol, isobutanol, t-butanol, n-pentanol, isopentanol, 2-nitroethanol, 2-fluoroethanol, 2,2,2-trifluoroethanol, ethylene glycol, 2-methoxyethanol, 1, 2-ethoxyethanol, diethylene glycol, 1, 2, or 3-pentanol, neo-pentyl alcohol, t-pentyl alcohol, diethylene glycol monoethyl ether, cyclohexanol, benzyl alcohol, phenol, or glycerol and the like; “polar solvents” such as water or mixtures thereof.

The term “suitable base” used herein the present invention until unless specified is selected from inorganic bases like “alkali metal hydroxides” such as lithium hydroxide, sodium hydroxide, potassium hydroxide and the like; “alkali metal carbonates” such as sodium carbonate, potassium carbonate, lithium carbonate and the like; “alkali metal bicarbonates” such as sodium bicarbonate, potassium bicarbonate, lithium bicarbonate and the like; “alkali metal hydrides” such as sodium hydride, potassium hydride, lithium hydride and the like; ammonia; and organic bases such as triethyl amine, methyl amine, ethyl amine, 1,8-diazabicyclo[5.4.0]undec-7-ene (DBU), 1,5-diazabicyclo(4.3.0)non-5-ene (DBN), lithium dioisoporpylamide (LDA), n-butyl lithium, tribenzylamine, isopropyl amine, diisopropylamine, diisopropylethylamine, N-methylmorpholine, N-ethylmorpholine, piperidine, dimethylaminopyridine, morpholine, pyridine, 2,6-lutidine, 2,4,6-collidine, imidazole, 1-methylimidazole, 1,2,4-triazole, 1,4-diazabicyclo[2.2.2]octane (DABCO); “alkali metal alkoxides” such as sodium methoxide, sodium ethoxide, sodium tert-butoxide, potassium methoxide, potassium ethoxide, potassium tert-butoxide and the like or mixtures thereof.

The suitable hydrochloric acid source is selected from HCl gas, aqueous HCl, dry HCl, ethyl acetate-HCl, isopropanolic HCl, ethanolic HCl, methanolic HCl, acetone HCl, acetonitrile-HCl and 1,3-dioxane-HCl.

As used herein the term “suitable acid” is selected from formic acid, acetic acid, propionic acid, butanoic acid, pentanoic acid, hexanoic acid, trifluoroacetic acid, methane sulfonic acid, HCl, HBr, HI and the like.

The need for protection and deprotection, and the selection of appropriate protecting groups can be readily determined by one skilled in the art. The chemistry of protecting groups can be found, for example, in Greene, et al., Protective Groups in Organic Synthesis, 4d. Ed., Wiley & Sons, 2007, which is incorporated herein by reference in its entirety.

In some embodiments, “hydroxy protecting group” is selected from benzyloxycarbonyl (Cbz), 2,2,2-trichloroethoxy carbonyl(Troc), 2-(trimethylsilyl)ethoxy carbonyl (Teoc), 2-(4-trifluoromethylphenylsulfonyl) ethoxycarbonyl (Tsc), t-butoxycarbonyl (BOC), 1-adamantyloxycarbonyl (Adoc), 2-adamantylcarbonyl(2-Adoc), 2,4-dimethylpent-3-yloxycarbonyl (Doc), cyclohexyloxy carbonyl(Hoc), 1,1-dimethyl-2,2,2-trichloroethoxycarbonyl (TcBoc), vinyl, 2-chloroethyl, 2-phenyl sulfonylethyl, allyl, benzyl, 2-nitrobenzyl, 4-nitrobenzyl, diphenyl-4-pyridylmethyl, N′,N′-dimethylhydrazinyl, methoxymethyl, t-butoxymethyl (Bum), benzyloxymethyl (BOM), or 2-tetrahydropyranyl (THP), 1-(ethoxy)ethyl, p-methoxybenzyl, triphenylmethyl, diphenylmethyl, hydroxymethyl, methoxymethyl, and t-butyldimethylsilylmethyl, N-pivaloyloxymethyl (POM), 1,1-diethoxymethyl, tri(C1-4-alkyl)silyl, p-methoxybenzyl carbonyl (Moz or MeOZ) group, 9-fluorenylmethyloxycarbonyl (FMOC) group, acetyl group, benzoyl (Bz) group, benzyl (Bn) group, p-methoxybenzyl (PMB), 3,4-dimethoxybenzyl.

According to some embodiments, the hydroxy protecting groups are selected from —C(O)OC₁-C₆ alkyl, such as, for example, carboethoxy, carbomethoxy and t-butoxycarbonyl; optionally substituted —C(O)OC₁-C₆ aryl, such as, for example, benzyloxy-carbonyl and p-methoxybenzyloxycarbonyl; optionally substituted —C₁-C₁₂ aryl(C₁-C₃)alkyl such as, for example, benzyl, phenethyl, p-methoxybenzyl, 2,3-dimethoxybenzyl, 2,4-dimethoxybenzyl and 9-fluorenylmethyl; optionally substituted C₇-C₁₁ aryl carbonyl, such as, for example, benzoyl; —C(O)R, wherein, R is an alkyl group having C₁-C₆ carbon atoms (For example acetyl or Ac or —C(O)CH₃, —C(O)CH₂CH₃, —C(O)CH₂CH₂CH₃ and the like); C₁-C₆ alkylsulfonyl, such as, for example, methanesulfonyl (mesyl).

The first aspect of the present invention provides a process for the preparation of D-glucitol, 1,5-anhydro-1-C-[4-chloro-3-[[4-[[(3S)-tetrahydro-3-furanyl]oxy] phenyl]methyl]phenyl]-, (1S) compound of formula-1

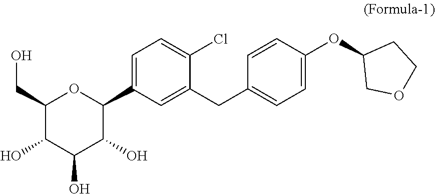 [SMILES: OC[C@H]1O[C@@H](c2ccc(Cl)c(Cc3ccc(O[C@H]4CCOC4)cc3)c2)[C@H](O)[C@@H](O)[C@@H]1O]

comprising, reacting the compound of general formula-3 with the compound of general formula-2 in presence of a suitable base in a suitable solvent.

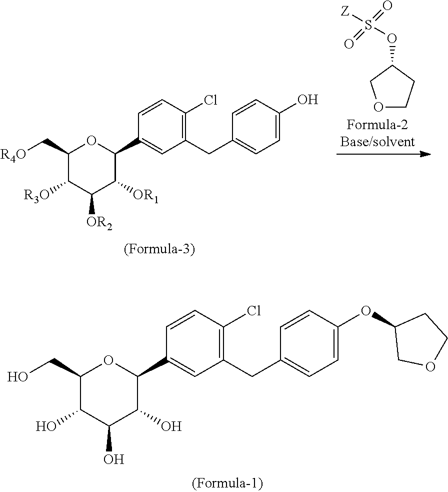

wherein, R₁, R₂, R₃ and R₄ are same or different selected from hydrogen or hydroxy protecting group selected from —C(O)R, —C(O)OR wherein R is an alkyl group having C₁-C₆ carbon atoms; optionally substituted —C(O)OC₁-C₆ aryl, optionally substituted —C₁-C₁₂ aryl(C₁-C₃)alkyl; optionally substituted C₇-C₁₁ aryl carbonyl; C₁-C₆ alkylsulfonyl; Z is alkyl group (C₁-C₄ carbon atoms) or aryl group (phenyl or naphthyl) substituted with one or more electron withdrawing groups such as —NO₂, —NH₃, —N(R₁)₃, —CN, —CHO, —COOH, trifluoroalkyl, halogen. Preferably, phenyl group substituted with —NO₂ in the ortho or para positions.

Most preferably, the compound of formula-2 is selected from (R)-tetrahydrofuran-3-yl 4-nitrobenzenesulfonate or (R)-tetrahydrofuran-3-yl 2-nitrobenzenesulfonate.

Wherein, the suitable base is selected from sodium hydroxide, sodium carbonate, sodium bicarbonate, potassium hydroxide, potassium carbonate, potassium bicarbonate, sodium hydride and the like; the suitable solvent is selected from alcohol solvents such as methanol, ethanol, n-propanol, isopropanol, n-butanol, isobutanol, t-butanol, n-pentanol, isopentanol, ethylene glycol and the like; chloro solvents such as dichloromethane, chloroform, carbon tetrachloride and the like; ketone solvents such as acetone, methyl ethyl ketone, pentanone and the like; polar aprotic solvents dimethylformamide, dimethylsulfoxide, dimethylacetamide, N-methylpyrrolidone; nitrile solvents such as acetonitrile, propionitrile and the like; ester solvents such as methyl acetate, ethyl acetate and propyl acetate and the like; hydrocarbon solvents such as n-hexane, n-heptane, benzene, toluene, o, m, p-xylene and the like; ether solvents such as diethyl ether, methyl tert-butyl ether, methyl ethyl ether, tetrahydrofuran and the like; and polar solvents like water or mixture thereof.

The preferred embodiment of the present invention provides a process for the preparation of D-glucitol, 1,5-anhydro-1-C-[4-chloro-3-[[4-[[(3S)-tetrahydro-3-furanyl]oxy]phenyl]methyl] phenyl]-, (1S) compound of formula-1 comprising: reacting the compound of general formula-3 with the compound of general formula-2 in presence of a suitable base in n-butanol solvent.

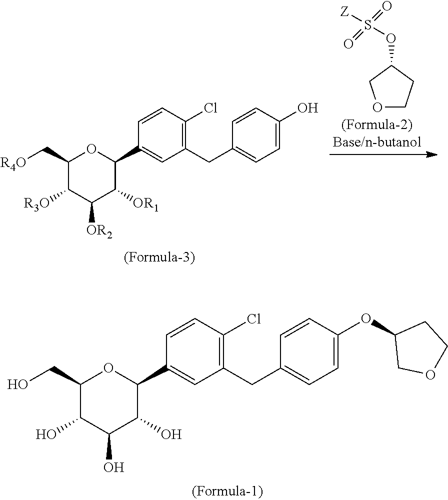

wherein, R₁ to R₄, Z and suitable base are same as defined hereinbefore.

Further, another preferred embodiment of the present invention provides a process for the preparation of D-glucitol, 1,5-anhydro-1-C-[4-chloro-3-[[4-[[(3S)-tetrahydro-3-furanyl]oxy] phenyl]methyl] phenyl]-, (1S) compound of formula-1, comprising: reacting the compound of general formula-3 with the compound of formula-2a in presence of potassium carbonate (K₂CO₃) in n-butanol solvent.

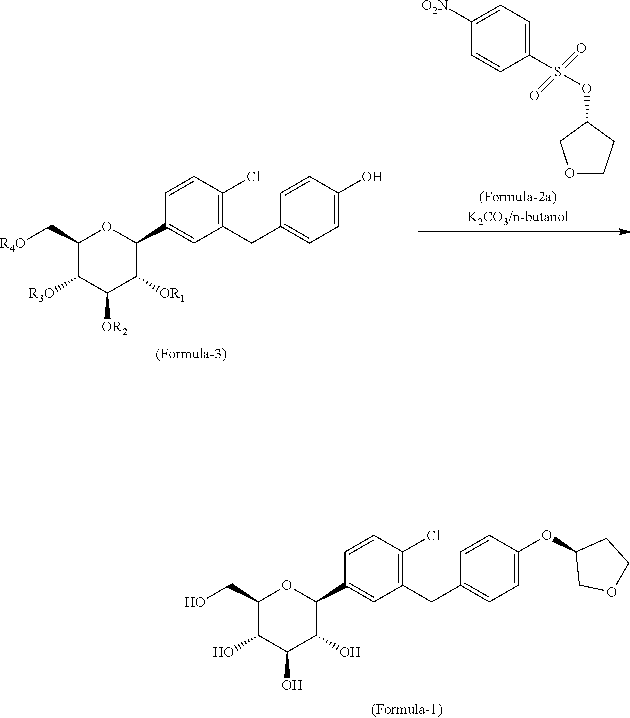

wherein, R₁ to R₄ are same as defined hereinbefore.

The another embodiment of the present invention provides a process for the preparation of D-glucitol, 1,5-anhydro-1-C-[4-chloro-3-[[4-[[(3S)-tetrahydro-3-furanyl]oxy]phenyl]methyl] phenyl]-, (1S) compound of formula-1, comprising:
a) reacting the compound of general formula-4 with suitable thiol reagent in presence of Lewis acid to provide compound of general formula-3;

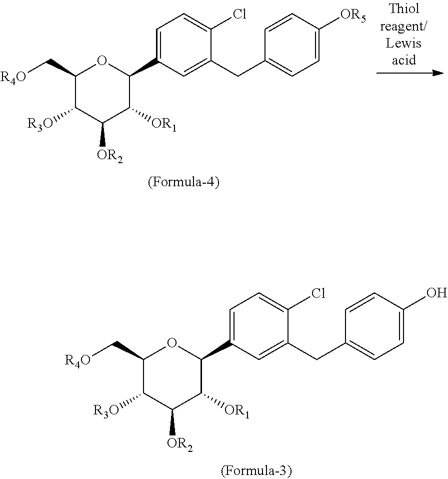

wherein, thiol reagent is selected from thiol or dithiol alcohol such as decanethiol, dodecane thiol, cyclohexane thiol, cyclopentane thiol, cyclo butane thiol, thiophenol, methanethiol, ethanethiol, 1-propanethiol, 2-propanethiol, n-butanethiol, tert-butanthiol, furan-2-ylmethanethiol, ethandithiol, 1,2-propanedithiol, 1,3-propanedithiol, 1,3-butanedithiol, 1,4-butanedithiol; suitable Lewis acid is selected from aluminium trihalides such as aluminum trichloride (AlCl₃), aluminum tribromide (AlBr₃) and the like, boron trihalides such as boron trichloride (BCl₃) and the like, titanium tetrachloride (TiCl₄), ferric chloride (FeCl₃), zinc chloride (ZnCl₂) and the like.
b) reacting of the compound of general formula-3 with the compound of general formula-2 in presence of a base in a solvent to provide D-glucitol, 1,5-anhydro-1-C-[4-chloro-3-[[4-[[(3S)-tetrahydro-3-furanyl]oxy] phenyl]methyl] phenyl]-, (1S) of formula-1

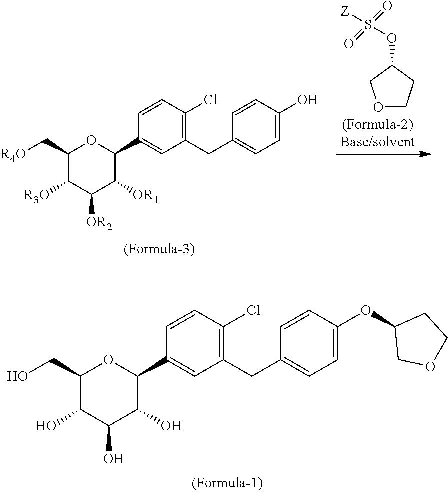

wherein, R₁ to R₄ are same as defined hereinbefore, R₅ is selected from an alkyl group having C₁-C₄ carbon atoms such as methyl, ethyl, n-propyl or isopropyl.

The preferred embodiment of the present invention provides a process for the preparation of D-glucitol, 1,5-anhydro-1-C-[4-chloro-3-[[4-[[(3S)-tetrahydro-3-furanyl]oxy]phenyl]methyl] phenyl]-, (1S) compound of formula-1, comprising:
a) reacting the compound of formula-4a with dodecanethiol in presence of aluminum trichloride (AlCl₃) to provide compound of formula-3a;

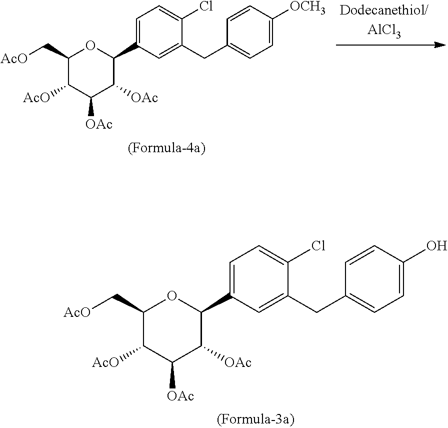 [SMILES: COc1ccc(Cc2cc([C@@H]3O[C@H](COC(C)=O)[C@@H](OC(C)=O)[C@H](OC(C)=O)[C@H]3OC(C)=O)ccc2Cl)cc1 ; CC(=O)OC[C@H]1O[C@@H](c2ccc(Cl)c(Cc3ccc(O)cc3)c2)[C@H](OC(C)=O)[C@@H](OC(C)=O)[C@@H]1OC(C)=O]

b) reacting the compound of formula-3a with (R)-tetrahydrofuran-3-yl 4-nitrobenzenesulfonate in presence of potassium carbonate in n-butanol to provide the compound of formula-1

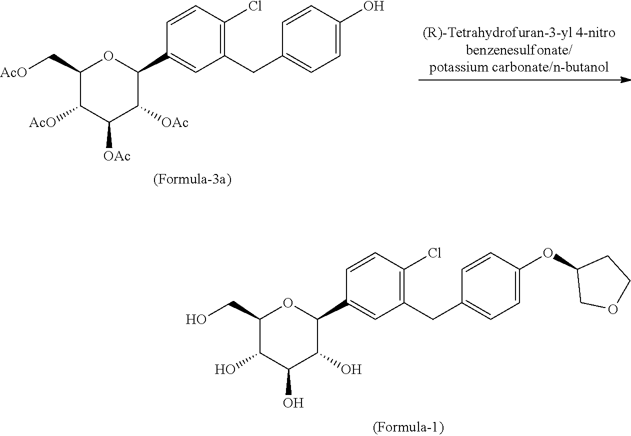 [SMILES: CC(=O)OC[C@H]1O[C@@H](c2ccc(Cl)c(Cc3ccc(O)cc3)c2)[C@H](OC(C)=O)[C@@H](OC(C)=O)[C@@H]1OC(C)=O ; OC[C@H]1O[C@@H](c2ccc(Cl)c(Cc3ccc(O[C@H]4CCOC4)cc3)c2)[C@H](O)[C@@H](O)[C@@H]1O]

The another embodiment of the present invention provides an improved process for the preparation of D-glucitol, 1,5-anhydro-1-C-[4-chloro-3-[[4-[[(3S)-tetrahydro-3-furanyl]oxy] phenyl]methyl] phenyl]-, (1S) compound of formula-1 comprising:
a) treating the compound of general formula-5 with suitable deacylating agent in a suitable solvent to provide the compound of general of formula-3;

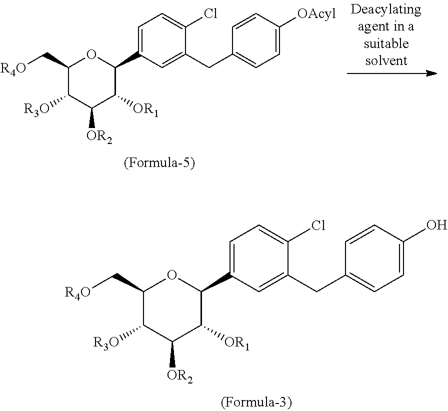

b) reacting of the compound of general formula-3 with the compound of general formula-2 in presence of a base in a solvent to provide D-glucitol, 1,5-anhydro-1-C-[4-chloro-3-[[4-[[(3S)-tetrahydro-3-furanyl]oxy] phenyl]methyl] phenyl]-, (1S) of formula-1

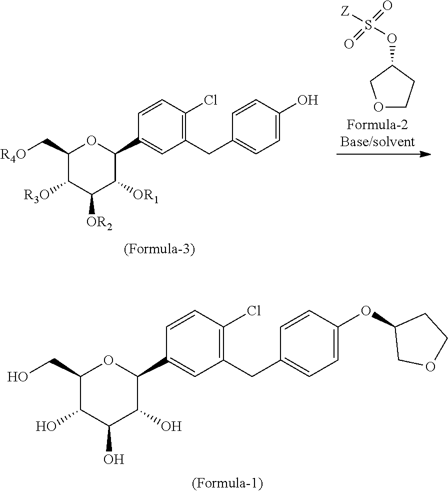

wherein, R₁ to R₄ and Z are same as defined hereinbefore.
wherein, in step-a), the acyl group is selected from —(CO)R₆, wherein R₆ is C₁-C₅ straight chain or branched substituted or unsubstituted alkyl groups, phenyl, benzyl and the like; the suitable deacylating agent is selected from ammonium acetate, sodium sulfite preferably ammonium acetate; in step-b), the suitable base is selected from sodium hydroxide, sodium carbonate, sodium bicarbonate, potassium hydroxide, potassium carbonate, potassium bicarbonate, sodium hydride and the like; the suitable solvent is selected from alcohol solvents such as methanol, ethanol, n-propanol, isopropanol, n-butanol, isobutanol, tert-butanol, n-pentanol, isopentanol, ethylene glycol and the like.

The preferred embodiment of the present invention provides a process for the preparation of D-glucitol, 1,5-anhydro-1-C-[4-chloro-3-[[4-[[(3S)-tetrahydro-3-furanyl]oxy]phenyl]methyl] phenyl]-, (1S) compound of formula-1, comprising:
a) treating the compound of formula-5a with ammonium acetate in methanol (MeOH) to provide the compound of formula-3a;

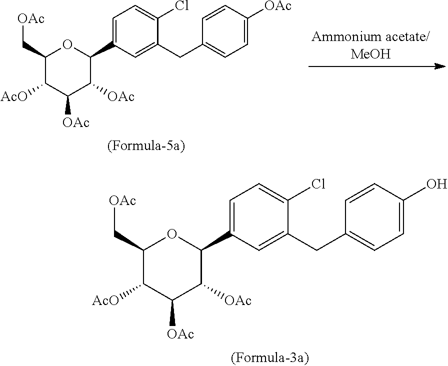 [SMILES: CC(=O)OC[C@H]1O[C@@H](c2ccc(Cl)c(Cc3ccc(OC(C)=O)cc3)c2)[C@H](OC(C)=O)[C@@H](OC(C)=O)[C@@H]1OC(C)=O ; CC(=O)OC[C@H]1O[C@@H](c2ccc(Cl)c(Cc3ccc(O)cc3)c2)[C@H](OC(C)=O)[C@@H](OC(C)=O)[C@@H]1OC(C)=O]

b) reacting of the compound of formula-3a with (R)-tetrahydrofuran-3-yl 4-nitrobenzenesulfonate in presence of potassium carbonate in n-butanol to provide D-glucitol, 1,5-anhydro-1-C-[4-chloro-3-[[4-[[(3S)-tetrahydro-3-furanyl]oxy] phenyl]methyl]phenyl]-, (1S) of formula-1

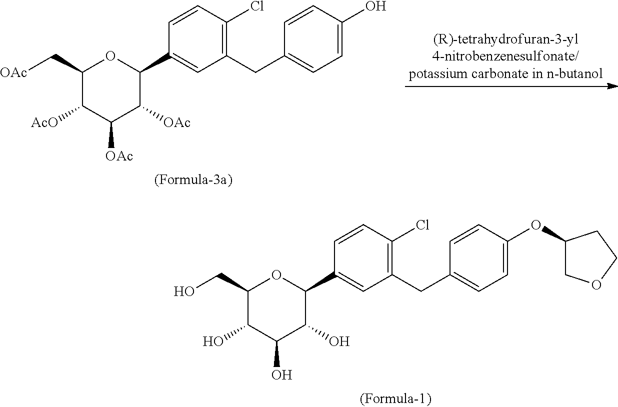 [SMILES: CC(=O)OC[C@H]1O[C@@H](c2ccc(Cl)c(Cc3ccc(O)cc3)c2)[C@H](OC(C)=O)[C@@H](OC(C)=O)[C@@H]1OC(C)=O ; OC[C@H]1O[C@@H](c2ccc(Cl)c(Cc3ccc(O[C@H]4CCOC4)cc3)c2)[C@H](O)[C@@H](O)[C@@H]1O]

In another embodiment of the present invention provides an improved process for the preparation of D-glucitol, 1,5-anhydro-1-C-[4-chloro-3-[[4-[[(3S)-tetrahydro-3-furanyl]oxy]phenyl] methyl] phenyl]-, (1S) compound of formula-1 comprising, reacting (2S,3R,4R,5S,6R)-2-(4-chloro-3-(4-hydroxybenzyl)phenyl)-6-(hydroxymethyl)tetrahydro-2H-pyran-3,4,5-triol of formula-6 with the compound of general formula-2 in presence of a base in a suitable solvent.

The process is schematically shown as below:

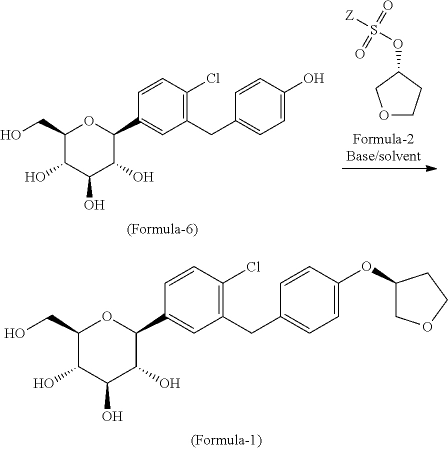

wherein, Z, base and solvent are same as defined hereinbefore.

In a preferred embodiment of the present invention provides an improved process for the preparation of pure D-glucitol, 1,5-anhydro-1-C-[4-chloro-3-[[4-[[(3S)-tetrahydro-3-furanyl]oxy] phenyl]methyl] phenyl]-, (1S) of formula-1, comprising: reacting (2S,3R,4R,5S,6R)-2-(4-chloro-3-(4-hydroxybenzyl)phenyl)-6-(hydroxymethyl)tetrahydro-2H-pyran-3,4,5-triol of formula-6 with (R)-tetrahydrofuran-3-yl 4-nitrobenzenesulfonate in presence of potassium carbonate in n-butanol.

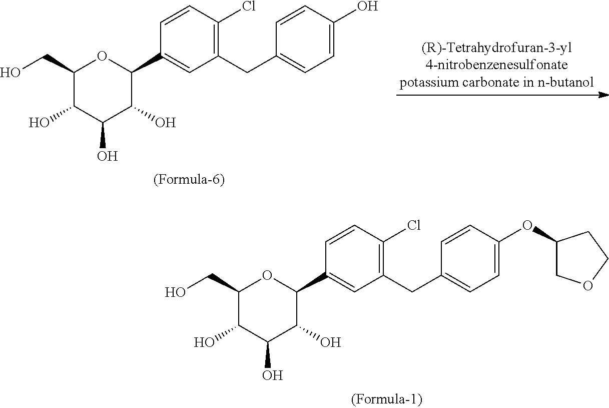 [SMILES: OC[C@H]1O[C@@H](c2ccc(Cl)c(Cc3ccc(O)cc3)c2)[C@H](O)[C@@H](O)[C@@H]1O ; OC[C@H]1O[C@@H](c2ccc(Cl)c(Cc3ccc(O[C@H]4CCOC4)cc3)c2)[C@H](O)[C@@H](O)[C@@H]1O]

The other embodiment of the present invention provides D-glucitol, 1,5-anhydro-1-C-[4-chloro-3-[[4-[[(3S)-tetrahydro-3-furanyl]oxy] phenyl]methyl]phenyl]-, (1S) of formula-1 which is obtained by the present invention can be further purified by crystallization or recrystallization using suitable solvents.

wherein, the suitable solvents are selected from alcohol solvents having C₁-C₄ carbon atoms such as methanol, ethanol, n-propanol, isopropanol, n-butanol and isobutanol; hydrocarbon solvents such as toluene, xylene, hexane, cyclohexane or their mixtures thereof.

The D-glucitol, 1,5-anhydro-1-C-[4-chloro-3-[[4-[[(3S)-tetrahydro-3-furanyl]oxy]phenyl]methyl]phenyl]-, (1S) formula-1 obtained by the present invention is having purity about more than 99% by HPLC and controls all the impurities below ICH limits.

The starting material of formula 4 can be prepared by the methods described in our PCT patent publication WO 2017/130217 A1.

The starting material of formula-5 can be prepared by the methods known in the prior art.

The starting material compound of formula-6 can be prepared by the methods known from U.S. Pat. No. 7,579,449 or any synthetic methods reported in prior art patents.

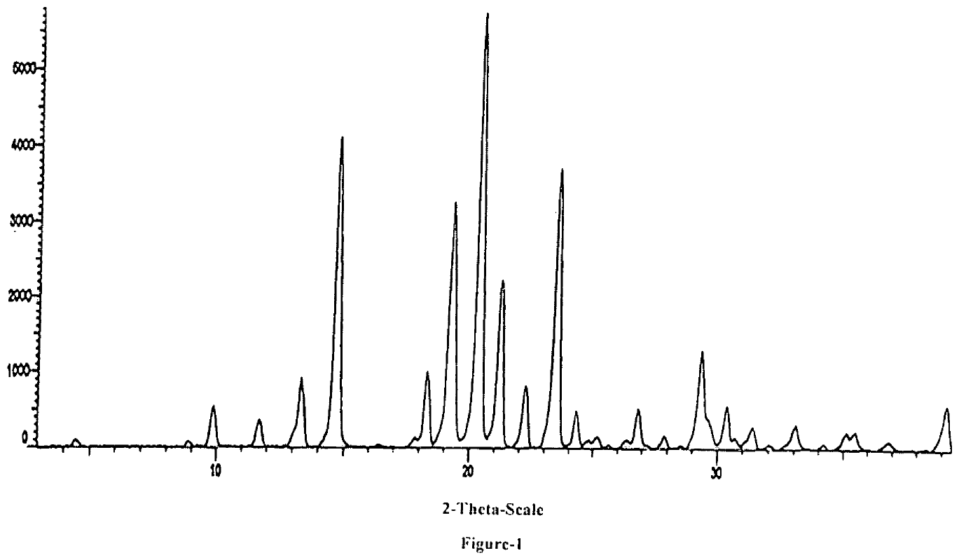

The second aspect of the present invention provides a crystalline form of D-glucitol, 1,5-anhydro-1-C-[4-chloro-3-[[4-[[(3S)-tetrahydro-3-furanyl]oxy] phenyl]methyl] phenyl]-, (1S) which is characterized by its PXRD pattern substantially in accordance with FIG. 1.

The third aspect of the present invention provides a process for the preparation of crystalline Form-S of D-glucitol, 1,5-anhydro-1-C-[4-chloro-3-[[4-[[(3S)-tetrahydro-3-furanyl]oxy] phenyl]methyl] phenyl]-, (1S), comprising:

a) dissolving Empagliflozin in a suitable solvent at a suitable temperature;

b) cooling the reaction mixture to a suitable temperature;

c) stirring the reaction mixture;

d) isolating crystalline Form-S of Empagliflozin.

wherein, in step-a), the suitable temperature is 35-100° C.; in step-b), the suitable temperature is 25-30° C.

The preferred embodiment of the present invention provides a process for the preparation of crystalline Form-S of Empagliflozin, comprising:

a) dissolving Empagliflozin in n-butanol solvent at 90° C. to 100° C.;

b) cooling the reaction mixture to 25° C. to 30° C.;

c) stirring the reaction mixture obtained in step-b);

d) isolating crystalline Form-S of Empagliflozin.

The fourth aspect of the present invention provides a process for the preparation of stable amorphous form of Empagliflozin, comprising:
a) adding Empagliflozin and pharmaceutically acceptable excipient to a suitable solvent;
b) heating the reaction mixture to a suitable temperature;
c) removing the solvent from the reaction mixture to provide an amorphous form.
   wherein, the ratio between Empagliflozin and pharmaceutically acceptable excipient is 1:0.01 to 0.05 (w/w).

The pharmaceutically acceptable excipients are selected from hydroxy propyl cellulose (HPC), cross-povidone, povidone (PVP K-30) and the like.

The other aspect of the present invention provides pharmaceutical compositions comprising a therapeutically effective amount of Empagliflozin one or more pharmaceutically acceptable carriers, excipients or diluents.

Pharmaceutical compositions containing crystalline Form-S of Empagliflozin and amorphous form of Empagliflozin of the present invention may be prepared by using diluents or excipients such as fillers, bulking agents, binders, wetting agents, disintegrating agents, surface active agents, and lubricants. Various modes of administration of the pharmaceutical compositions of the invention can be selected depending on the therapeutic purpose, for example tablets, pills, powders, liquids, suspensions, emulsions, granules, capsules, suppositories, or injection preparations.

The oral pharmaceutical composition may contain one or more additional excipients such as diluents, binders, disintegrants and lubricants. Exemplary diluents include lactose, sucrose, glucose, mannitol, sorbitol, calcium carbonate, microcrystalline cellulose, magnesium stearate and mixtures thereof. Exemplary binders are selected from L-hydroxy propyl cellulose, povidone, hydroxypropyl methyl cellulose, hydroxylethyl cellulose and pre-gelatinized starch.

Exemplary disintegrants are selected from croscarmellose sodium, cros-povidone, sodium starch glycolate and low substituted hydroxylpropyl cellulose.

Exemplary lubricants are selected from sodium stearyl fumarate, magnesium stearate, zinc stearate, calcium stearate, stearic acid, talc, glyceryl behenate and colloidal silicon dioxide. A specific lubricant is selected from magnesium stearate, zinc stearate, calcium stearate and colloidal silicon dioxide.

PXRD analysis of crystalline Empagliflozin was carried out using BRUKER-AXS D8 Advance X-Ray diffractometer using Cu-Ka radiation of wavelength 1.5406 A° and at continuous scan speed of 0.03°/min.

The HPLC analysis of the present invention was analyzed by HPLC under the following conditions:

Apparatus: A liquid chromatographic system equipped with variable wavelength UV detector; Column: Cosmicsil PN, 250*4.6 mm, 5 μm (or) equivalent; Column temperature: 25° C.; Wave length: 225 nm; Injection volume: 10 μL; Elution: Gradient; Diluent: acetonitrile:water (90:10) % v/v; Buffer: weigh accurately about 2.72 gm of potassium dihydrogen phosphate in 1000 mL of Milli-Q-water. Filter this solution through 0.22 μm Nylon membrane filter paper and sonicate to degas; Mobile phase-A: Buffer: acetonitrile:methanol (70:10:20)% v/v/v Mobile phase-B: Acetonitrile:methanol:water (60:20:20)% v/v/v.

The best mode of carrying out the present invention is illustrated by the below mentioned examples. These examples are for illustrative purposes only and in no way limit the embodiments of the present invention.

### EXAMPLES

### Example-1: Preparation of the (2R, 3R, 4R, 5S, 6S)-2-(acetoxymethyl)-6-(4-chloro-3-(4-hydroxybenzyl)phenyl)tetrahydro-2H-pyran-3,4,5-triyl triacetate of Formula-3a

Aluminum chloride (94.85 gm) and dichloromethane (200 ml) were charged into a round bottom flask at 25-30° C. Dodecanethiol (143 gms) was added to the above resulted reaction mixture at 25-30° C. and cooled the reaction mixture to 15-20° C. To this reaction mixture, a solution of (2R,3R,4R,5S,6S)-2-(acetoxymethyl)-6-(4-chloro-3-(4-methoxybenzyl) phenyl) tetrahydro-2H-pyran-3,4,5-triyl triacetate of formula-4a (100 gms) in 200 ml of dichloromethane was added at 15-20° C. and stirred for 3 hrs at 25-30° C. Chilled water (1000 ml) was added to the above reaction mixture and stirred for about 15 mins at 25-30° C. Separated the both organic and aqueous layers. The aqueous layer was extracted with dichloromethane. Combined the total organic layers was washed with aqueous sodium carbonate solution and again washed with water. Distilled off the solvent completely from the organic layer under reduced pressure and co-distilled with cyclohexane. Cyclohexane (600 ml) was added to the above distillate and heated the reaction mixture to 45-50° C. and stirred for 45 min. at same temperature. Cooled the reaction mixture to 25-30° C. and stirred for 2 hrs. Filtered the material, washed with cyclohexane and then dried to get the title compound. (Yield: 86.0 gms)

### Example-2: Preparation of the (2R, 3R, 4R, 5S, 6S)-2-(acetoxymethyl)-6-(4-chloro-3-(4-hydroxybenzyl) phenyl)tetrahydro-2H-pyran-3,4,5-triyl triacetate of Formula-3a

Aluminum chloride (25.8 gms) was added to dichloromethane (75 ml) in a round bottom flask at 25-30° C. Dodecanethiol (125 gms) was added to the above reaction mixture at 25-30° C. To this reaction mixture, (2R,3R,4R,5S,6S)-2-(acetoxymethyl)-6-(4-chloro-3-(4-ethoxybenzyl)phenyl)tetrahydro-2H-pyran-3,4,5-triyl triacetate solution (25 gms in 50 ml of dichloromethane) was added at 25-30° C. and stirred for 3 hrs at same temperature. Water (250 ml) was added to the above reaction mixture and stirred for about 15 mins. Separated the organic, aqueous layers and washed the organic layer with water. Distilled off the solvent completely from the organic layer under reduced pressure and then co-distilled with cyclohexane. Cyclohexane (375 ml) was added to the obtained distillate at 40-50° C. and cooled the reaction mixture to 25-30° C. and stirred for 2 hrs. Filtered the obtained compound and washed with cyclohexane. The obtained compound was dried to get the title compound. (Yield: 20.0 gms)

### Example-3: Preparation of the (2R, 3R, 4R, 5S, 6S)-2-(acetoxymethyl)-6-(4-chloro-3-(4-hydroxybenzyl) phenyl)tetrahydro-2H-pyran-3,4,5-triyl triacetate of formula-3a

(2R,3R,4R,5S,6S)-2-(Acetoxymethyl)-6-(4-chloro-3-(4-ethoxybenzyl)phenyl)tetra-hydro-2H-pyran-3,4,5-triyl triacetate (105 gms) was added to dichloromethane (630 ml) at 25-30° C. Cooled the reaction mixture to 10-15° C. and added dodecanethiol (170.1 gms) and then stirred for 10 min. Aluminium chloride (112.5 gms) was slowly added lot wise to the above reaction mixture and stirred for about 15 mins. Raised the temperature of the reaction mixture to 25-30° C. and stirred for 6 hrs. Added the above obtained reaction mixture to chilled water (1050 ml) at 0-10° C. Washed the reaction mixture with dichloromethane and separated the organic, aqueous layers. Washed the organic layer with aqueous sodium bicarbonate solution (26.3 gms in 525 ml). Separated the organic and aqueous layers and distilled off the solvent completely from the organic layer under reduced pressure and then co-distilled with cyclohexane. Cyclohexane (630 ml) was added to the obtained distillate at 50-55° C. and stirred for 45 min and cooled the reaction mixture to 25-30° C. and stirred for 90 min. Filtered the obtained compound, washed with cyclohexane and then dried to afford the title compound. (Yield: 90.6 gms)

### Example-4: Preparation of the (2R, 3R, 4R, 5S, 6S)-2-(acetoxymethyl)-6-(4-chloro-3-(4-hydroxybenzyl) phenyl)tetrahydro-2H-pyran-3,4,5-triyl triacetate of Formula-3a

(2S,3S,4R,5R,6R)-2-(3-(4-Acetoxybenzyl)-4-(chlorophenyl)-6-(acetoxymethyl)tetra-hydro-2H-pyran-3,4,5-triyl triacetate (15 gms) was added to methanol (45 ml) at 25-30° C. Ammonium acetate (15.6 gms) and tetrahydrofuran (90 ml) were added to the above reaction mixture at 25-30° and stirred for 10 mins. Raised the temperature of the reaction mixture to 60-65° C. and stirred for 5 hrs at same temperature. Water was added to the obtained reaction mixture and cooled the temperature to 25-30° C. Separated the organic, aqueous layers and extracted the aqueous layer with ethyl acetate. Distilled off the solvent completely from the organic layer under reduced pressure. Cyclohexane (105 ml) was added to the obtained material at 60-65° C. and stirred for 3 hrs at same temperature. Filtered the precipitated solid, washed with cyclohexane and then dried to afford the title compound. (Yield: 12.3 gms)

### Example-5: Preparation of (R)-tetrahydrofuran-3-yl 4-nitrobenzenesulfonate

(R)-3-Hydroxyfuran (100 gms) and dichloromethane (1000 ml) were charged into a clean and dry RBF at 25-30° C. and stirred for 10 min at same temperature. To the resulted reaction mixture, dimethylaminopyridine (11.8 gms) and triethylamine (229 gms) were added and cooled to 0-5° C. p-Nitro benzenesulfonyl chloride (301.0 gms) was added to the above reaction mixture and raised the temperature to 25-30° C. and stirred for 4 hrs at same temperature. Water (500 ml) was added to the above reaction mixture and stirred for 20 min. and separate the aqueous and organic layers. The organic layer was washed with aqueous hydrochloride solution and followed by with aqueous sodium bicarbonate solution. Distilled off the solvent completely from the organic layer and co-distilled with methanol. The obtained compound was dissolved in methanol (500 ml) at 60-65° C. and stirred for 1 hr. Cooled the reaction mixture to 0-5° C. and again stirred for 2 hrs. Filtered the precipitated solid, washed with methanol and then dried to get the title compound as a solid. (Yield: 280.0 gms).

### Example-6: D-glucitol, 1,5-anhydro-1-C-[4-chloro-3-[[4-[[(3S)-tetrahydro-3furanyl]oxy] phenyl]methyl] phenyl]-, (1S) (Empagliflozin (Formula-1))

(2R, 3R,4R, 5S,6S)-2-(Acetoxymethyl)-6-(4-chloro-3-(4-hydroxybenzyl)phenyl)tetra hydro-2H-pyran-3,4,5-triyl triacetate (100 gms), (R)-tetrahydrofuran-3-yl 4-nitrobenzene sulfonate (54.7 gms) and n-butanol (1000 ml) were mixed in a clean and dry round bottom flask at 25-30° C. To this reaction mixture, potassium carbonate (125.8 gms) was added and heated the reaction mixture to 80° C. and stirred for 2 hrs. Filtered the reaction mixture and washed with n-butanol and distilled off the solvent completely from the filtrate under reduced pressure. Cooled the reaction mixture to 25-30° C. and then added ethyl acetate and water. Heated the reaction mixture to 45-50° C. and stirred for 45 mins at same temperature. Separated the organic and aqueous layers. Extracted the aqueous layer with ethyl acetate and combined the total organic layers. Distilled off the solvent completely from the organic layer under reduced pressure. Ethyl acetate (500 ml) was added to the above obtained residue compound at 25-30° C. Heated the reaction mixture to 70° C. and stirred for 60 min at same temperature. Cooled the reaction mixture to 25-30° C. and stirred for 2 hours at same temperature. Filtered the obtained material and washed with ethyl acetate and then dried to get the solid compound. Dissolved the obtained solid in the mixture of methanol (100 ml) and toluene (100 ml) at 65-70° C. Cooled the reaction mixture to 25-30° C. and further to 0-5° C. and stirred for 2 hrs at 0-5° C. Filtered the obtained solid, washed with methanol and toluene mixture and then dried to get title compound. (Yield: 55 gms, M.R: 165-170)

### Example-7: Preparation of (3R,4S,5R,6R)-3,4,5-tris((trimethylsilyl)oxy)-6-(((trimethyl-silyl)oxy)methyl)tetrahydro-2H-pyran-2-one

N-Methyl morpholine (NMM) (127.85 gms) was added to the solution of (3R,4S,5S,6R)-3,4,5-trihydroxy-6-(hydroxymethyl)tetrahydro-2H-pyran-2-one (25 gms) in tetrahydrofuran (250 ml) at 25-30° C. Cooled the reaction mixture to 0-5° C. and slowly added trimethylsilyl chloride (144 gms) for 3 hrs. Heated the reaction mixture to 45-50° C. and stirred for 12 hrs at same temperature. Cooled the reaction mixture to 10-15° C. and added toluene (125 ml), chilled water (125 ml). Raised the temperature of reaction mixture to 25-30° C. and stirred for 15 min. Separated the both organic, aqueous layers and extracted the aqueous layer with toluene. Combined the both organic layers and washed with 10% aqueous sodium dihydrogen phosphate solution. Separated the organic and aqueous layers and washed the organic layer with 10% aqueous sodium chloride solution. Distilled off the solvent completely from the organic layer under reduced pressure and co-distilled with toluene to get the title compound (Yield: 66.25 gms).

### Example-8: Preparation of (2S,3R,4S,5S,6R)-2-(4-chloro-3-(4-methoxybenzyl)phenyl)-6-(hydroxymethyl)-2-methoxytetrahydro-2H-pyran-3,4,5-triol

Dissolved 4-bromo-1-chloro-2-(4-methoxybenzyl)benzene (25 gms) in tetrahydrofuran (150 ml) and toluene (50 ml) at 25-30° C. Cooled the reaction mixture to −75° C. to −80° C. and stirred for 20 min at same temperature under nitrogen atmosphere. Slowly added n-BuLi (15% n-hexane) (85.5 ml) to the above reaction mixture at −75° C. to −80° C. under nitrogen atmosphere and stirred for 30 min at same temperature. (3R, 4S, 5R, 6R)-3,4,5-Tris ((trimethylsilyl)oxy)-6-(((trimethylsilyl)oxy)methyl)tetrahydro-2H-pyran-2-one (67.4 gms) in toluene (25 ml) was slowly added to the above reaction mixture at −75° C. to −80° C. and stirred for 90 min at same temperature. Slowly added methanesulfonic acid (23 gms) in methanol (150 ml) to the above reaction mixture at −75° C. to −80° C. and stirred for 30 min at same temperature. Raised the temperature of the reaction mixture to 25-30° C. and stirred for 17 hrs at same temperature. Washed the reaction mixture with water and separated both aqueous and organic layers. Water was added to the organic layer and stirred for 15 min at 25-30° C. Separated the both organic and aqueous layers. The aqueous layer was basified with aqueous sodium carbonate solution. Ethyl acetate was added to the above reaction mixture and stirred for 20 min. Separated the both aqueous and organic layers and extracted the aqueous layer with ethyl acetate. Combined the total organic layers and washed with aqueous sodium chloride solution. Charcoal (5 gms) was added to the organic layer and stirred for 15 min. Filtered the reaction mixture through hyflow bed and washed with ethyl acetate. Distilled off the solvent completely from the filtrate under reduced pressure and co-distilled with toluene. The above obtained compound was taken in 75 ml of toluene and slowly added to n-hexane and stirred for 60 min at 25-30° C. Filtered the obtained compound and washed with n-hexane under nitrogen atmosphere. n-Hexane (75 ml) was added to the above filtrate and stirred for 45 min at 25-30° C. Filtered the obtained compound, washed with n-hexane and then dried to get the title compound. (Yield: 40 gms, Melting Range: 50-55° C.)

### Example-9: Preparation of D-glucitol, 1,5-anhydro-1-C-[4-chloro-3-[[4-[[(3S)-tetrahydro-3-furanyl]oxy]phenyl]methyl]phenyl]-, (1S) (Empagliflozin (Formula-1))

(2S, 3R, 4R, 5S, 6R)-2-(4-Chloro-3-(4-hydroxybenzyl)phenyl)-6-(hydroxymethyl) tetrahydro-2H-pyran-3,4,5-triol (100 gms), (R)-tetrahydrofuran-3-yl 4-nitrobenzene sulfonate (79.0 gms) and n-butanol (1000 ml) were mixed in a clean and dry round bottom flask at 25-30° C. Potassium carbonate (181.7 gms) was added to the above reaction mixture at 25-30° C. Heated the reaction mixture to 80-85° C. and stirred for 2 hrs at same temperature. Filtered the reaction mixture and washed with n-butanol. Distilled-off the solvent completely from the above filtrate under reduced pressure. Ethyl acetate and water were added to the obtained compound at 25-30°. Heated the reaction mixture to 45-50° C. and stirred for 45 mins at same temperature. Separated the organic and aqueous layers. Extracted the aqueous layer with ethyl acetate and combined the total organic layers. Distilled off the solvent completely from the organic layer under reduced pressure. Ethyl acetate was added to the above residue compound at 25-30° C. Heated the reaction mixture to 70° C. and stirred for 60 min at same temperature. Cooled the reaction mixture to 25-30° C. and stirred for 2 hours at same temperature. Filtered the solid material and washed with ethyl acetate. The obtained compound was recrystallized in methanol to get the pure crystalline title compound. (Yield: 35.0 gms, MR: 165-170° C.)

### Example-10: Preparation of D-glucitol, 1,5-anhydro-1-C-[4-chloro-3-[[4-[[(3S)-tetrahydro-3-furanyl]oxy]phenyl]methyl]phenyl]-, (1S) (Empagliflozin (Formula-1))

(2S,3R,4R,5S,6R)-2-(4-Chloro-3-(4-hydroxybenzyl)phenyl)-6-(hydroxymethyl)tetra-hydro-2H-pyran-3,4,5-triol (90.0 gms) was added to n-butanol (900 ml) and potassium carbonate (113.3 gms) at 25-30° C. and stirred for 10 min at same temperature. (R)-Tetrahydrofuran-3-yl 4-nitrobenzene sulfonate (55.9 gms) was added to the reaction mixture at 25-30° C. Heated the reaction mixture to 75-80° C. and stirred for 3 hrs at same temperature and cooled to 60-65° C. Filtered the obtained compound and washed with n-butanol. Distilled off the solvent completely from the filtrate under reduced pressure. Dissolved the obtained material in methanol (270 ml) at 25-30° C. and added water (1620 ml) to the reaction mixture and stirred for 2 hrs at same temperature. Filtered the obtained compound and washed with water. Methanol (270 ml) was added to the above material at 25-30° C. and raised the temperature of reaction mixture to 60-65° C. and stirred for 1 hr. Filtered the reaction mixture and washed with methanol. Distilled off the solvent completely from the filtrate under reduced pressure and co-distilled with ethyl acetate. Ethyl acetate (270 ml) was added to the obtained material at 25-30° C. and raised the temperature of the reaction mixture to 60-65° C. and stirred for 30 min. Cooled the reaction mixture to 25-30° C. and stirred for 2 hrs. Filtered the material and washed with ethyl acetate. Methanol (135 ml) was added to the above material at 25-30° C. and raised the temperature of the reaction mixture to 60-65° C. and stirred for 30 min. Cooled the reaction mixture to 0-5° C. and stirred for 2 hrs. Filtered the precipitated solid, washed with methanol and then dried to get the title compound. (Yield: 32.0 gms, purity by HPLC: 99.89%, R-isomer: 0.07%) The PXRD pattern of the obtained compound was similar to the PXRD pattern disclosed in U.S. Pat. No. 7,713,938.

### Example-11: Preparation of Crystalline Form-S of D-glucitol, 1,5-anhydro-1-C-[4-chloro-3-[[4-[[(3S)-tetrahydro-3-furanyl]oxy] phenyl] methyl] phenyl]-, (1S)

D-Glucitol, 1,5-anhydro-1-C-[4-chloro-3-[[4-[[(3S)-tetrahydro-3-furanyl]oxy]phenyl]methyl]phenyl]-, (1S) (500 mg) was dissolved in n-butanol (30 ml) at 95-100° C. Cooled the reaction mixture to 25-30° C. and stirred for 20 hours at same temperature. Filtered the obtained solid and then dried to get the title compound. (Yield: 420 mg) The PXRD of the obtained compound is illustrated in FIG. 1.

### Example-12: Preparation of Stable Amorphous Form of Empagliflozin

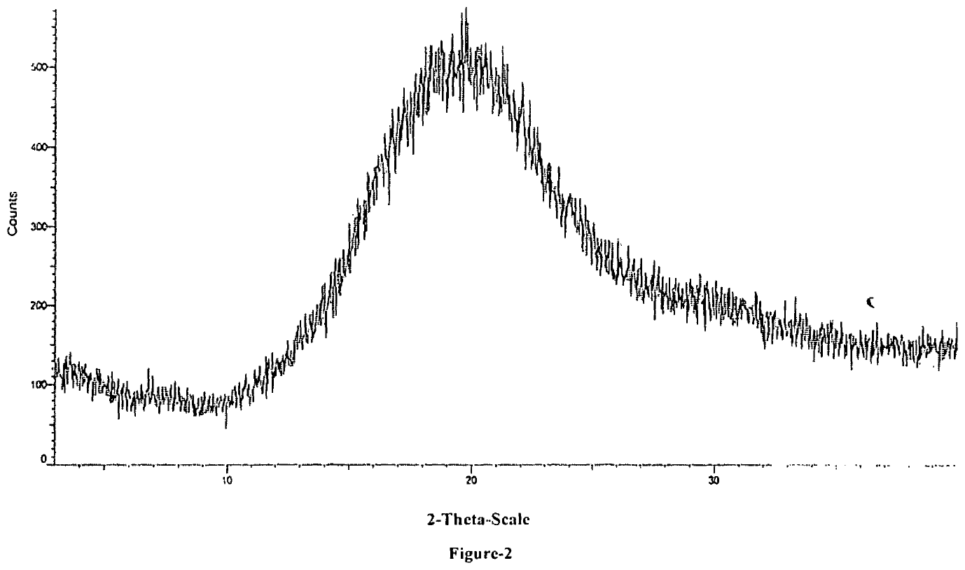

Empagliflozin (10 gms) dissolved in methanol (300 ml) at 60-65° C. Filtered the reaction mixture and distilled off the solvent completely from the filtrate under reduced pressure and then dried to get the title compound. (Yield: 8.5 gms, Purity by HPLC: 99.6%) The PXRD of the obtained compound is illustrated in FIG. 2.

### Example-13: Preparation of Stable Amorphous Form of Empagliflozin

Empagliflozin (10 gms) was dissolved in methanol (300 ml) at 60-65° C. polyvinylpyrrolidone (PVP-K30) (10.0 mg) was added to the above reaction mixture at 60-65° C. and stirred for 10 min at same temperature. Filtered the reaction mixture and distilled off the solvent completely from the filtrate under reduced pressure and then dried to get the title compound. (Yield: 8.8 gms, Purity by HPLC: 99.69%) The PXRD of the obtained compound is similar to FIG. 2.

### Example-14: Preparation of Stable Amorphous Form of Empagliflozin

Empagliflozin (10 gms) and cross-povidone (10 gms) were dissolved in methanol (500 ml) at 60-65° C. Filtered the obtained reaction mixture and distilled off the solvent completely from the filtrate under reduced pressure and then dried to get the title compound. (Yield: 9.4 gms). The PXRD of the obtained compound is similar to FIG. 2.

### Example-15: Preparation of Stable Amorphous Form of Empagliflozin

Empagliflozin (10 gms) was dissolved in methanol (300 ml) at 60-65° C. Polyvinylpyrrolidone (PVP-K30) (50.0 mg) was added to the reaction mixture at 60-65° C. and stirred for 10 min at same temperature. Filtered the reaction mixture and distilled off the solvent completely from the filtrate under reduced pressure and then dried to get the title compound. (Yield: 8.6 gms, Purity by HPLC: 99.42%)

The PXRD of the obtained compound is illustrated in FIG. 2.

1. A process for the preparation of D-glucitol, 1,5-anhydro-1-C-[4-chloro-3-[[4-[[(3S)-tetrahydro-3-furanyl]oxy] phenyl]methyl] phenyl]-, (1S) compound of formula-1, 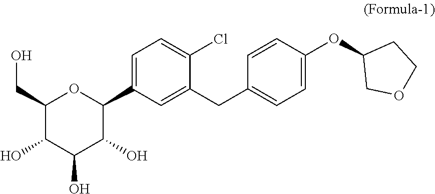 [SMILES: OC[C@H]1O[C@@H](c2ccc(Cl)c(Cc3ccc(O[C@H]4CCOC4)cc3)c2)[C@H](O)[C@@H](O)[C@@H]1O]
comprising of reacting the compound of general formula-3 with the compound of general formula-2 in presence of a suitable base in a suitable solvent 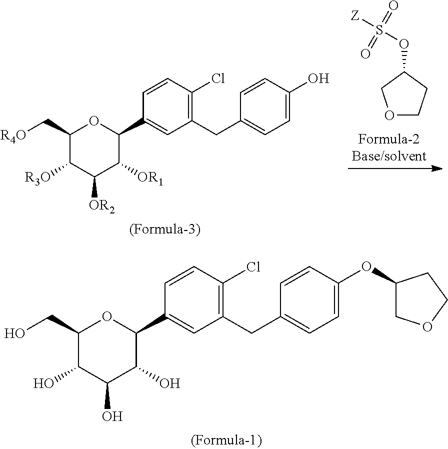
wherein, R₁, R₂, R₃ and R₄ are refers to hydrogen or hydroxy protecting group which can be selected from —C(O)R, wherein, R is an alkyl group having C₁-C₆ carbon atoms, —C(O)OC₁-C₆ alkyl, optionally substituted —C(O)OC₁-C₆ aryl, optionally substituted —C₁-C₁₂ aryl(C₁-C₃)alkyl, optionally substituted C₇-C₁₁ aryl carbonyl, C₁-C₆ alkylsulfonyl; Z is alkyl group (C₁-C₄ carbon atoms) or aryl group (phenyl or naphthyl) substituted with one or more electron withdrawing groups such as —NO₂, —NH₃, —N(R₁)₃, —CN, —CHO, —COOH, trifluoroalkyl, halogen.

2. The process according to claim 1, wherein the suitable base is selected from sodium hydroxide, sodium carbonate, sodium bicarbonate, potassium hydroxide, potassium carbonate, potassium bicarbonate, sodium hydride and the like; the suitable solvent is selected from alcohol solvents such as methanol, ethanol, n-propanol, isopropanol, n-butanol, isobutanol, t-butanol, n-pentanol, isopentanol, ethylene glycol or mixtures.

3. A process for the preparation of D-glucitol, 1,5-anhydro-1-C-[4-chloro-3-[[4-[[(3S)-tetrahydro-3-furanyl]oxy] phenyl]methyl] phenyl]-, (1S) compound of formula-1 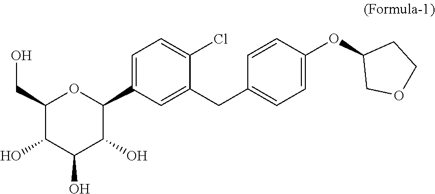 [SMILES: OC[C@H]1O[C@@H](c2ccc(Cl)c(Cc3ccc(O[C@H]4CCOC4)cc3)c2)[C@H](O)[C@@H](O)[C@@H]1O]
comprising of reacting the compound of general formula-3 with the compound of general formula-2 in presence of a suitable base in n-butanol 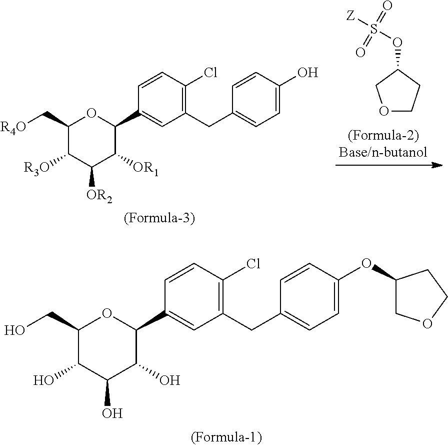
wherein, R₁, R₂, R₃ and R₄ are same or different selected from hydrogen or hydroxy protecting group which can be selected from —C(O)R, wherein, R is an alkyl group having C₁-C₆ carbon atoms, —C(O)OC₁-C₆ alkyl, optionally substituted —C(O)OC₁-C₆ aryl, optionally substituted —C₁-C₁₂ aryl(C₁-C₃)alkyl, optionally substituted C₇-C₁₁ aryl carbonyl, C₁-C₆ alkylsulfonyl; Z is alkyl group (C₁-C₄ carbon atoms) or aryl group (phenyl or naphthyl) substituted with one or more electron withdrawing groups such as —NO₂, —NH₃⁺, —N(R₁)₃, —CN, —CHO, —COOH, trifluoroalkyl, halogen.

4. The process according to claim 3, wherein, the suitable base is selected from sodium hydroxide, sodium carbonate, sodium bicarbonate, potassium hydroxide, potassium carbonate, potassium bicarbonate, sodium hydride and the like.

5. A process for the preparation of D-glucitol, 1,5-anhydro-1-C-[4-chloro-3-[[4-[[(3S)-tetrahydro-3-furanyl]oxy] phenyl]methyl] phenyl]-, (1S) compound of formula-1 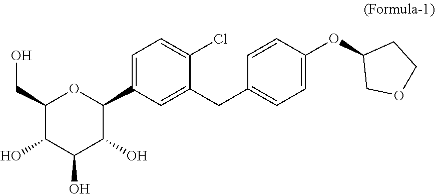 [SMILES: OC[C@H]1O[C@@H](c2ccc(Cl)c(Cc3ccc(O[C@H]4CCOC4)cc3)c2)[C@H](O)[C@@H](O)[C@@H]1O]
comprising reacting the compound of general formula-3 with the compound of formula-2a in presence of potassium carbonate in n-butanol 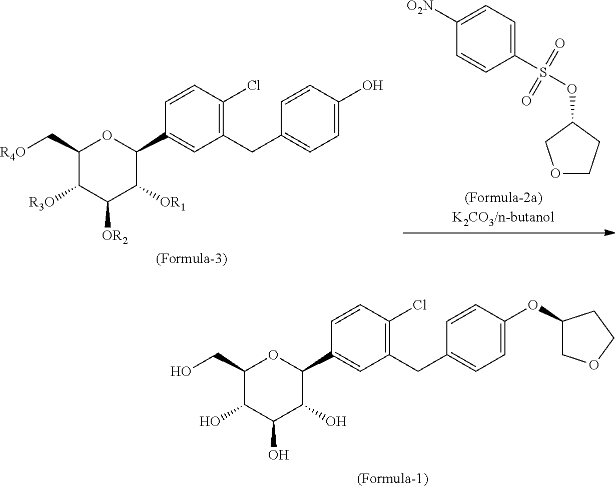
wherein, R₁, R₂, R₃ and R₄ are same or different selected from hydrogen or hydroxy protecting group which can be selected from —C(O)R, wherein, R is an alkyl group having C₁-C₆ carbon atoms, —C(O)OC₁-C₆ alkyl, optionally substituted —C(O)OC₁-C₆ aryl, optionally substituted —C₁-C₁₂ aryl(C₁-C₃)alkyl, optionally substituted C₇-C₁₁ aryl carbonyl, C₁-C₆ alkylsulfonyl.

6. A process for the preparation of D-glucitol, 1,5-anhydro-1-C-[4-chloro-3-[[4-[[(3S)-tetrahydro-3-furanyl]oxy] phenyl]methyl] phenyl]-, (1S) compound of formula-1 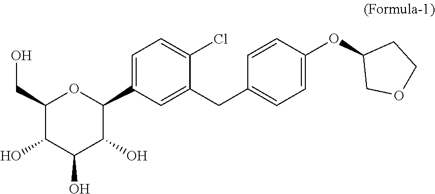 [SMILES: OC[C@H]1O[C@@H](c2ccc(Cl)c(Cc3ccc(O[C@H]4CCOC4)cc3)c2)[C@H](O)[C@@H](O)[C@@H]1O]
comprising:
a) reacting the compound of general formula-4 with suitable thiol reagent in presence of Lewis acid to provide the compound of general formula-3; 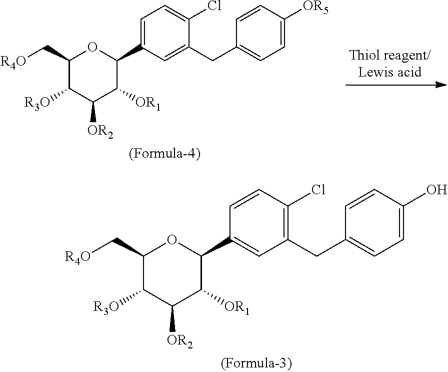
b) reacting of the compound of general formula-3 with the compound of general formula-2 in presence of a base in a solvent to provide D-glucitol, 1,5-anhydro-1-C-[4-chloro-3-[[4-[[(3S)-tetrahydro-3-furanyl]oxy] phenyl]methyl] phenyl]-, (1S) of formula-1 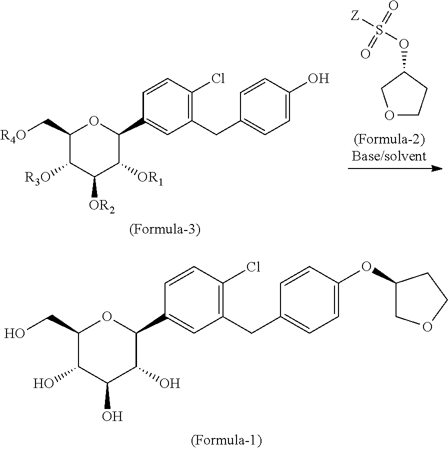
wherein, R₁, R₂, R₃ and R₄ are refers to hydrogen or hydroxy protecting group selected from —C(O)R, wherein, R is an alkyl group having C₁-C₆ carbon atoms, —C(O)OC₁-C₆ alkyl, optionally substituted —C(O)OC₁-C₆ aryl, optionally substituted —C₁-C₁₂ aryl(C₁-C₃)alkyl, optionally substituted C₇-C₁₁ aryl carbonyl, C₁-C₆ alkylsulfonyl, methanesulfonyl, trialkylsilyl. R₅ is selected from an alkyl group having C₁-C₄ carbon atoms such as methyl, ethyl, n-propyl or isopropyl; Z is alkyl group (C₁-C₄ carbon atoms) or aryl group (phenyl or naphthyl) substituted with one or more electron withdrawing groups such as —NO₂, —NH₃, —N(R₁)₃, —CN, —CHO, —COOH, trifluoroalkyl, halogen.

7. A process for the preparation of D-glucitol, 1,5-anhydro-1-C-[4-chloro-3-[[4-[[(3S)-tetrahydro-3-furanyl]oxy] phenyl]methyl] phenyl]-, (1S) compound of formula-1 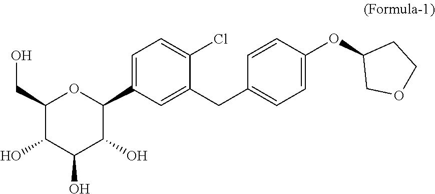 [SMILES: OC[C@H]1O[C@@H](c2ccc(Cl)c(Cc3ccc(O[C@H]4CCOC4)cc3)c2)[C@H](O)[C@@H](O)[C@@H]1O]
comprising the steps of:
a) reacting the compound of formula-4a with dodecanethiol in presence of aluminum trichloride to provide the compound of formula-3a; 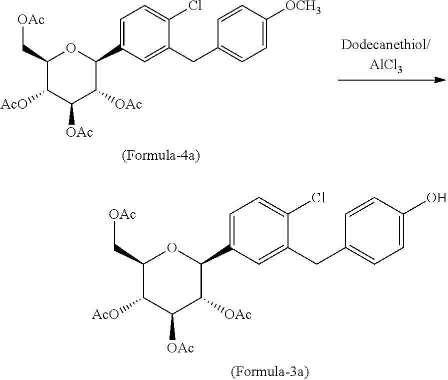 [SMILES: COc1ccc(Cc2cc([C@@H]3O[C@H](COC(C)=O)[C@@H](OC(C)=O)[C@H](OC(C)=O)[C@H]3OC(C)=O)ccc2Cl)cc1 ; CC(=O)OC[C@H]1O[C@@H](c2ccc(Cl)c(Cc3ccc(O)cc3)c2)[C@H](OC(C)=O)[C@@H](OC(C)=O)[C@@H]1OC(C)=O]
b) reacting the compound of formula-3a with (R)-tetrahydrofuran-3-yl 4-nitrobenzenesulfonate in presence of potassium carbonate in n-butanol to provide the compound of formula-1

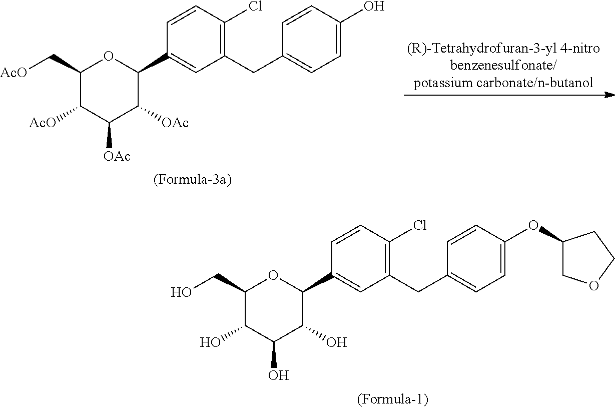 [SMILES: CC(=O)OC[C@H]1O[C@@H](c2ccc(Cl)c(Cc3ccc(O)cc3)c2)[C@H](OC(C)=O)[C@@H](OC(C)=O)[C@@H]1OC(C)=O ; OC[C@H]1O[C@@H](c2ccc(Cl)c(Cc3ccc(O[C@H]4CCOC4)cc3)c2)[C@H](O)[C@@H](O)[C@@H]1O]

8. A process for the preparation of D-glucitol, 1,5-anhydro-1-C-[4-chloro-3-[[4-[[(3S)-tetrahydro-3-furanyl]oxy]phenyl]methyl] phenyl]-, (1S) compound of formula-1, comprising:
a) treating the compound of formula-5a with ammonium acetate in methanol to provide the compound of formula-3a; 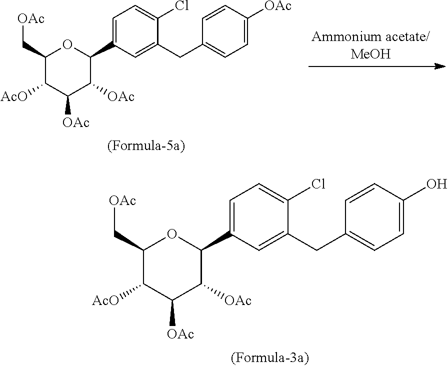 [SMILES: CC(=O)OC[C@H]1O[C@@H](c2ccc(Cl)c(Cc3ccc(OC(C)=O)cc3)c2)[C@H](OC(C)=O)[C@@H](OC(C)=O)[C@@H]1OC(C)=O ; CC(=O)OC[C@H]1O[C@@H](c2ccc(Cl)c(Cc3ccc(O)cc3)c2)[C@H](OC(C)=O)[C@@H](OC(C)=O)[C@@H]1OC(C)=O]
b) reacting of the compound of formula-3a with (R)-tetrahydrofuran-3-yl 4-nitrobenzenesulfonate in presence of potassium carbonate in n-butanol to provide D-glucitol, 1,5-anhydro-1-C-[4-chloro-3-[[4-[[(3S)-tetrahydro-3-furanyl]oxy] phenyl]methyl]phenyl]-, (1S) of formula-1

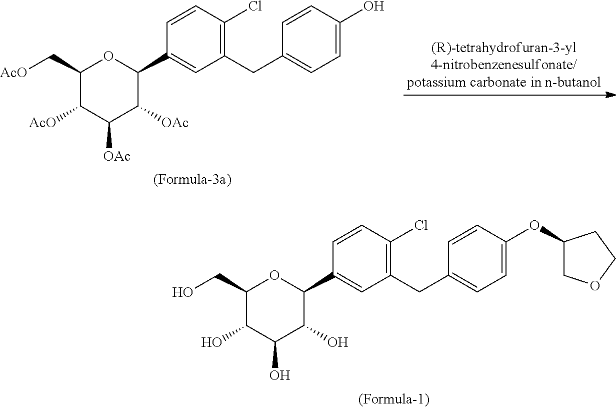 [SMILES: CC(=O)OC[C@H]1O[C@@H](c2ccc(Cl)c(Cc3ccc(O)cc3)c2)[C@H](OC(C)=O)[C@@H](OC(C)=O)[C@@H]1OC(C)=O ; OC[C@H]1O[C@@H](c2ccc(Cl)c(Cc3ccc(O[C@H]4CCOC4)cc3)c2)[C@H](O)[C@@H](O)[C@@H]1O]

9. A process for the preparation of D-glucitol, 1,5-anhydro-1-C-[4-chloro-3-[[4-[[(3S)-tetrahydro-3-furanyl]oxy]phenyl] methyl] phenyl]-, (1S) compound of formula-1 comprising, reacting (2S,3R,4R, 5S,6R)-2-(4-chloro-3-(4-hydroxybenzyl)phenyl)-6-(hydroxymethyl)tetrahydro-2H-pyran-3,4,5-triol of formula-6 with the compound of formula-2 in presence of a base in a suitable solvent 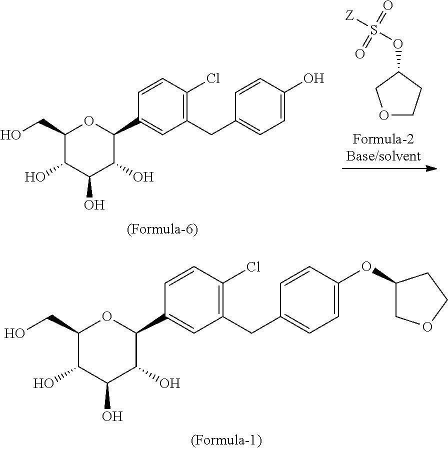
wherein, Z is alkyl group (C₁-C₄ carbon atoms) or aryl group (phenyl or naphthyl) substituted with one or more electron withdrawing groups such as —NO₂, —NH₃, —N(R₁)₃, —CN, —CHO, —COOH, trifluoroalkyl, halogen.

10. The process according to claim 9, the suitable base is selected from alkali metal hydroxides, alkali metal carbonates, alkali metal bicarbonates and the like; the suitable solvent is selected from alcohol solvents, chloro solvents, ketone solvents, polar aprotic solvents, nitrile solvents, ester solvents, hydrocarbon solvents, ether solvents and polar solvents like water or mixture thereof.

11. A process for the preparation of pure D-glucitol, 1,5-anhydro-1-C-[4-chloro-3-[[4-[[(3S)-tetrahydro-3-furanyl]oxy] phenyl]methyl] phenyl]-, (1S) of formula-1, comprising: reacting (2S,3R,4R, 5S,6R)-2-(4-chloro-3-(4-hydroxybenzyl)phenyl)-6-(hydroxymethyl) tetrahydro-2H-pyran-3,4,5-triol of formula-6 with (R)-tetrahydrofuran-3-yl 4-nitrobenzenesulfonate in presence of potassium carbonate in n-butanol.

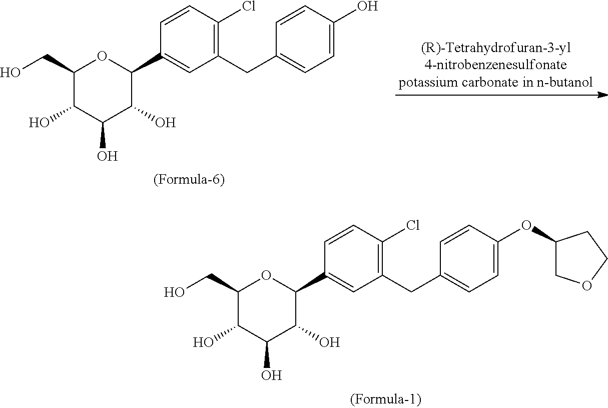 [SMILES: OC[C@H]1O[C@@H](c2ccc(Cl)c(Cc3ccc(O)cc3)c2)[C@H](O)[C@@H](O)[C@@H]1O ; OC[C@H]1O[C@@H](c2ccc(Cl)c(Cc3ccc(O[C@H]4CCOC4)cc3)c2)[C@H](O)[C@@H](O)[C@@H]1O]

12. Use of n-butanol in the condensation of compound of general formula-2 and compound of general formula-3.

13. A process for the preparation of D-glucitol, 1,5-anhydro-1-C-[4-chloro-3-[[4-[[(3S)-tetrahydro-3-furanyl]oxy] phenyl]methyl] phenyl]-, (1S) compound of formula-1, comprising reaction of the compound of general formula-3 with the compound of general formula-2 in presence of a suitable solvent.
